(19) United States
(12) Patent Application Publication
(10) Pub. No.: US 2025/0381133 A1
(43) Pub. Date: Dec. 18, 2025

(54) ARTICLE IN THE FORM OF EDIBLE SHEET

(71) Applicant: TECOZYME INC, Seoul (KR)

(72) Inventor: Yoon NAM, Seoul (KR)

(73) Assignee: TECOZYME INC, Seoul (KR)

(21) Appl. No.: 19/315,384

(22) Filed: Aug. 29, 2025

Related U.S. Application Data
(63) Continuation-in-part of application No. 17/596,646, filed on Dec. 15, 2021.

(30) Foreign Application Priority Data
Jul. 24, 2020 (KR) 10-2020-0092654

(51) Int. Cl. A61K 9/00; A61K 31/353; A61K 33/04; A61K 33/06; A61K 33/26; A61K 33/30; A61K 33/32; A61K 33/34; A61K 47/36

(52) U.S. Cl. CPC A61K 9/006; A61K 31/353; A61K 33/04; A61K 33/06; A61K 33/26; A61K 33/30; A61K 33/32; A61K 33/34; A61K 47/36

(57) ABSTRACT

The present disclosure relates to an article in the form of an edible sheet. The edible sheet of the present disclosure can be processed into a thin form such as a sheet, has excellent adhesiveness to the mucous membrane in the oral cavity, has excellent durability, and is decomposed at an appropriate rate by moisture in the oral cavity, and can be used for various purposes.

### CROSS-REFERENCE TO RELATED APPLICATIONS

This application is a continuation-in-part of U.S. application Ser. No. 17/596,646, filed May 25, 2021, which is a national stage application under 35 U.S.C. § 371 of International Application No. PCT/KR2021/006495, filed May 25, 2021, which claims the benefit of priority to Korean Patent Application No. 10-2020-0092654, filed Jul. 24, 2020, the entire disclosures of which are incorporated herein by reference.

### TECHNICAL FIELD

The present disclosure relates to an article in the form of an edible sheet.

### DESCRIPTION OF RELATE ART

Hyaluronic acid is a natural substance synthesized in the human body, and particularly, it is known that it is abundant in the skin. It is known to constitute the dermis together with an elastin protein tissue or a collagen protein tissue in the skin in a gel-phase under general conditions.

Since such hyaluronic acid contains many hydroxy groups in terms of molecular structure, it has very high hydrophilicity, water retention, and water-bearing properties, and thus it is known to have a moisturizing or absorbent action that supplies moisture to the skin from within the skin.

In addition, as it is a material that is bio-synthesized in the human body, it is known to be very safe for human use, and is widely used as an additive in cosmetics, pharmaceuticals, or eye drops.

Recently, a lot of health functional foods and general foods in particular for the purpose of directly ingesting hyaluronic acid itself have been released, and many applications are being attempted in the form of hydrogels, freeze-dried foams, and the like.

However, such hyaluronic acid is a polysaccharide-type polymer material in which a portion of the hydroxy group is substituted with an amide bond. Since it exists as a unique gel phase under general conditions or conditions in the human body, it is very difficult to process it into a thin form such as a sheet.

Meanwhile, green tea catechin, a polyphenol compound known for its antioxidant, antibacterial, and anti-inflammatory properties, suffer from a short retention time in the oral mucosa and rapid oxidation, significantly limiting their therapeutic efficacy. Therefore, innovative drug delivery strategies are needed to enhance stability and extend their duration of action in the oral cavity.

### SUMMARY

The present specification provides an edible sheet processed into a thin form such as a sheet including hyaluronic acid.

The present specification provides an edible sheet including hyaluronic acid, hypromellose and green tea catechin.

The hyaluronic acid may include one or more of a cross-linked hyaluronic acid and a non-cross-linked hyaluronic acid.

In addition, the hypromellose may preferably have a methoxy substitution rate of about 10 to about 35%, or about 20 to about 25%, and a hydroxypropyl substitution rate of about 1to about 15%, about 3 to about 15%, or about 5 to about 10%.

According to an embodiment of the invention, the edible sheet may preferably have a dry thickness of about 0.05 to about 1 mm, or about 0.1 to about 0.5 mm. In this connection, the drying may be performed by a drying method for about 18 to about 30 hours under conditions of about 25 to about 45° C., preferably for about 18 to about 30 hours at a temperature condition similar to human body temperature, or a rapid drying method at a high temperature of about 80 to about 120° C. for about 10 to about 60 minutes.

In addition, the edible sheet may include about 0.1 to about 20 parts by weight of a hyaluronic acid, based on 100 parts by weight of hypromellose. The lower limit of the content of a hyaluronic acid may be about 0.1 parts by weight or more, preferably about 1 part by weight or more, or about 5 parts by weight or more, and the upper limit of the content of a hyaluronic acid may be about 20 parts by weight or less, or about 16 parts by weight or less.

In another aspect, the edible sheet may include about 3 to about 7 parts by weight of a green tea catechin, based on 100 parts by weight of hypromellose.

In another aspect, the edible sheet prevents oxidation of green tea catechin.

In another aspect, the edible sheet is air-dried.

HPMC/HA/GTC encapsulation system enabled the preparation of thin, slowly air-dried patches without premature oxidation of catechins, providing a significantly more cost-effective alternative to freeze-drying methods. In addition, the hyaluronic acid may preferably have a weight average molecular weight of about 10,000 to about 1,000,000 g/mol, about 10,000 to about 100,000 g/mol, or about 10,000 to about 50,000 g/mol.

In addition, the hypromellose may preferably have a weight average molecular weight of 100,000 to 1,000,000 g/mol or about 100,000 to about 500,000 g/mol.

According to another embodiment of the invention, the edible sheet may further include ions of one or more edible metals selected from the group consisting of zinc, copper, iron, nickel, manganese, chromium, calcium, magnesium, sodium, potassium and selenium, in addition to hyaluronic acid and hypromellose.

In this connection, the hyaluronic acid and the hypromellose may be in the form of a complex coordinated around the metal, in other words, in the form of a metal coordination compound.

In addition, in this connection, the ion of the metal, the ion of the metal, may be included in a ratio of 0.01 to 2 moles with respect to a total of 100 g of the hyaluronic acid and the hypromellose, preferably a ratio of 0.1 to 0.5 moles with respect to a total of 100 g of the hyaluronic acid and the hypromellose, or a ratio of 0.15 to 0.35 moles with respect to a total of 100 g of the hyaluronic acid and the hypromellose.

The edible sheet according to an embodiment of the present disclosure i) may have a form that additionally includes a material other than hyaluronic acid, hypromellose and green tea catechin, or ii) does not contain a material other than hyaluronic acid, hypromellose and green tea catechin, and may consist only of a hyaluronic acid, hypromellose and green tea catechin.

Terms used herein are used to describe exemplary embodiments and are not intended to limit the scope of the present disclosure.

The terms of a singular form may include plural forms unless otherwise specified.

As used herein, the terms “comprising,” “including” or “having,” or the like are used to specify the presence of stated features, integers, steps, components, or a combination thereof, but do not preclude the presence or addition of one or more other features, integers, steps, components, or combinations thereof.

In addition, as used herein, each layer or element is referred to as being formed “on” or “over” respective layers or elements, which means that each layer or element may be formed directly on respective layers or elements or another layer or element may be additionally formed between respective layers or on a target material or substrate.

The present disclosure may be variously modified and have various types, and specific embodiments of the present disclosure will be descried below in detail with reference to the accompanying drawing. However, the present disclosure is not limited to the exemplary embodiments described herein, but all of the modifications, equivalents, and substitutions within the spirit and scope of the present disclosure are also included in the present disclosure.

Hereinafter, the present disclosure will be described in detail.

According to one aspect of the present disclosure, there is provided an edible sheet including hyaluronic acid, hypromellose and green tea catechin.

The inventors of the present disclosure completed the present disclosure based on the fact that when only hyaluronic acid is used alone, due to its unique gel form, it is very difficult to process it in a film or sheet form, and even when processed in a thin film or sheet form, it is easily decomposed in the oral cavity when ingested, its form disintegrates immediately, whereas when hyaluronic acid and hypromellose are used together, it can be processed relatively easily into a thin film or sheet form, and even when ingested, its form is maintained to some extent in the oral cavity. In addition, green tea catechin are used together, the polymeric encapsulation system by using the hyaluronic acid enabled the preparation of thin, slowly air-dried patches without premature oxidation of catechins, providing a significantly more cost-effective alternative to freeze-drying methods.

The hyaluronic acid may include one or more of cross-linked hyaluronic acid and non-cross-linked hyaluronic acid.

The cross-linked hyaluronic acid means that in non-cross-linked hyaluronic acid in the form of a polysaccharide linear polymer in which a portion of the hydroxy group is substituted with an amide bond, a crosslinking bond is formed between each linear polymer.

Such cross-linked hyaluronic acid has established in vivo safety as much as non-cross-linked hyaluronic acid, and due to its unique cross-linked structure, when cross-linked hyaluronic acid and non-cross-linked hyaluronic acid are used simultaneously, it is possible to control the rate of dissolution in the oral cavity when ingested.

Hypromellose is a substance in which a portion of the hydroxy group present in the cellulose polymer is substituted with a methoxy group or a hydroxypropoxy group, and is also called hydroxypropyl methyl cellulose.

Hypromellose is in the form of a pale yellow to white powder or granule, and when water is added thereto, it increases in volume and has a mucus form.

Hypromellose also has established safety for the human body to some extent, and due to the above properties or the influence of hydroxy groups present a lot in molecules, it is widely used as an emulsifier, thickener, suspension stabilizer, etc., and is also used as food coating ingredients.

For such hypromellose, the physical properties of the polymer may vary depending on the degree of substitution of a methoxy group or a hydroxypropoxy group in the hydroxy group originally present in the cellulose polymer. When considering compatibility with hyaluronic acid, processability when processing in a sheet form, rate of decomposition in the oral cavity when ingested, and degree of decomposition, it may be preferable to use those having a methoxy substitution rate of about 10 to about 35%, or about 20 to about 25%, and a hydroxypropyl substitution rate of about 3 to about 158, or about 5 to about 10%.

Meanwhile, the green tea catechin according to one embodiment of the present invention contained epicatechin (EC), epigallocatechin (EGC), gallate epicatechin (ECG), and epigallocatechin-3-gallate (EGCG). Hydrogen bonding between the hydroxyl group (—OH) of HPMC and the gallol group of EGCG is a partially cross-linked network.

Upon saliva exposure, the gallol group of green tea catechin interacts with the HPMC framework and mucin, rapidly converting them into numerous nano-sized nanogels (nanoparticles, nanospheres). These nanogels exhibit strong adhesion to the oral mucosa due to their high surface-to-volume ratio and are widely distributed throughout the oral cavity. This conversion promotes the sustained release of catechins and minimizes oral foreign body sensation, thereby enhancing user convenience.

As described above, both hyaluronic acid and hypromellose are polymers in which repeating units of monosaccharides in the form of hexagonal rings are long linked, and have very similar molecular structures and have excellent compatibility because there are many hydroxy groups in the molecule.

In the case of using hyaluronic acid and hypromellose together, according to the above-described technical principle, the disadvantages of using each of a hyaluronic acid and hypromellose alone can be effectively compensated. In particular, when processed into a thin form such as a sheet or film, its durability or adhesion degree attached to the mucous membrane in the oral cavity is greatly increased.

In addition, in the case of using hyaluronic acid, hypromellose and green tea catechin together, according to the above-described technical principle, the disadvantages of using each of a hyaluronic acid, hypromellose and green tea catechin alone can be effectively compensated. In particular, the polymeric encapsulation system by using the hyaluronic acid enabled the preparation of thin, slowly air-dried patches without premature oxidation of catechins, providing a significantly more cost-effective alternative to freeze-drying methods.

In other words, the sheet or food in a film form containing hyaluronic acid, hypromellose and green tea catechin is not easily decomposed in the oral cavity when ingested, and when it is attached to the oral mucosa in the oral cavity, it is not easily detached from the attachment site.

In consideration of the above characteristics, the edible sheet according to an embodiment of the present disclosure may be used as a simple edible film, and may also be used as a patch attached to a specific site in the oral cavity. When used for such a use as a patch, the film or

sheet slowly disintegrates in the oral cavity by including pharmaceuticals of specific ingredients, sweeteners, various vitamins, and/or various functional substances having an anti-odor function in the film or sheet, and may be used for the purpose of maintaining the oral use uniformly for a specific period of time. Alternatively, by attaching the film or sheet to the mucous membrane in the oral cavity, it may be used for uniform absorption into the mucous membrane for a specific period of time.

In addition, in this connection, the above-described functional substances such as pharmaceutical ingredients and sweeteners are not contained in the edible sheet itself of the present disclosure, but are contained in a kind of patch bag made of the edible sheet, or may be made in the form of being additionally coated or added to the top of the surface of the edible sheet of the present disclosure.

According to an embodiment of the invention, the edible sheet may preferably have a dry thickness of about 0.05 to about 1 mm, or about 0.1 to about 0.5 mm. In this connection, the drying may be performed by a drying method for about 18 to about 30 hours under conditions of about 25 to about 45° C., preferably for about 18 to about 30 hours at a temperature condition similar to human body temperature, or a rapid drying method at a high temperature of about 80 to about 120° C. for about 10 to about 60 minutes.

When the dry thickness of the edible sheet is in the above range, it is possible to maintain adequate strength in the oral cavity when food is ingested, and while the shape is released over time, it may properly perform the role as a patch or film.

In addition, the edible sheet may include about 0.1 to about 20 parts by weight of a hyaluronic acid, and about 3 to about 7 parts by weight of a green tea catechin based on 100 parts by weight of hypromellose. The lower limit of the content of a hyaluronic acid may be about 0.1 parts by weight or more, preferably about 1 part by weight or more, or about 5 parts by weight or more, and the upper limit of the content of a hyaluronic acid may be about 20 parts by weight or less, or about 16 parts by weight or less.

In the edible sheet according to an embodiment of the present disclosure, when the relative content of a hyaluronic acid and hypromellose is in the above range, the mutual complementary effect of the above-described hyaluronic acid and hypromellose can be maximized. In particular, adhesiveness and durability to mucous membranes in the oral cavity can be greatly improved.

In particular, there is always a lot of moisture in the oral cavity by saliva, and stimulation may be constantly applied by the tongue, teeth, cheeks, etc. In this aspect, when the relative content of a hyaluronic acid is too much or too little, the adhesiveness of the edible sheet in the oral cavity is lowered, or the durability of the sheet is lowered.

In addition, the hyaluronic acid may preferably have a weight average molecular weight of about 10,000 to about 1,000,000 g/mol, about 10,000 to about 100,000 g/mol, or about 10,000 to about 50,000 g/mol.

In addition, the hypromellose may preferably have a weight average molecular weight of 100,000 to 1,000,000 g/mol or about 100,000 to about 500,000 g/mol.

When the weight average molecular weight of a hyaluronic acid and hypromellose is within the above range, the above-described interaction and complementary effects of a hyaluronic acid and hypromellose can be maximized. In particular, adhesiveness to mucous membranes in the oral cavity, durability, and degradability over time can be appropriately adjusted.

According to another embodiment of the invention, the edible sheet may further include ions of one or more edible metals selected from the group consisting of zinc, copper, iron, nickel, manganese, chromium, calcium, magnesium, sodium, potassium and selenium, in addition to hyaluronic acid and hypromellose.

The above-mentioned ions of metals are generally present in a polycation state. It is possible to further improve the adhesiveness and durability of the edible sheet in the oral cavity by electrostatic interaction with numerous hydroxy groups present in hyaluronic acid and hypromellose contained in the edible sheet of the present disclosure.

In this connection, the hyaluronic acid and the hypromellose may be in the form of a complex coordinated around the metal, that is, in the form of a metal coordination compound.

In addition, the above-mentioned metals are mineral components helpful to the human body, and when such edible metals are included, the edible sheet according to an embodiment of the present disclosure may be used as health supplements.

In addition, in this connection, the ion of the metal, the ion of the metal, may be included in a ratio of 0.01 to 2 moles with respect to a total of 100 g of the hyaluronic acid and the hypromellose, preferably a ratio of 0.1 to 0.5 moles with respect to a total of 100 g of the hyaluronic acid and the hypromellose, or a ratio of 0.15 to 0.35 moles with respect to a total of 100 g of the hyaluronic acid and the hypromellose.

However, the present disclosure is not necessarily limited to the above range, and the content of these metals may vary depending on the above-described electrostatic interaction and recommended daily intake for each metal.

When the content of metal ions is too small, the above-mentioned beneficial effect may not be implemented well. When the content of metal ions is too high, the amount of absorption of metal ions into the human body increases, resulting in side effects. In addition, the electrostatic interaction with numerous hydroxy groups present in hyaluronic acid and hypromellose becomes too large, so that the edible sheet according to an embodiment of the present disclosure is not properly decomposed in the oral cavity.

In other words, the edible sheet according to an embodiment of the present disclosure i) may have a form that additionally includes a material other than hyaluronic acid, hypromellose and green tea catechin, or ii) does not contain a material other than hyaluronic acid, hypromellose and green tea catechin, and may consist of only a hyaluronic acid, hypromellose and green tea catechin.

In the case of an edible sheet composed only of a hyaluronic acid and hypromellose, it may be used simply as a snack food. In addition, as described above, the edible sheet may be used as a form containing various additives or functional substances in a kind of patch bag made of an edible sheet, other various additives or functional materials may be used in the form of being additionally coated or added on the surface of the edible sheet.

Examples of such additives include natural or synthetic sweeteners such as xylitol, menthol, aspartame, sodium saccharin, sucralose, sorbitol, acesulfame potassium, neopharm, stevia, and polydextrose; medicinal ingredients of naturally-derived extracts such as green tea, black tea, ginseng, and red ginseng such as catechins, saponins, and ginsenosides; and additives for formulation such as glycerin.

The edible sheet of the present disclosure may be prepared by the following method.

First, hyaluronic acid and hypromellose are dissolved in an appropriate amount of purified water. In this case, the purified water is preferably used in an amount of about 2 to about 10 times the total weight of the hyaluronic acid and hypromellose. In addition, for uniform dispersion of a hyaluronic acid and hypromellose at this time, it may be preferable to maintain the temperature of the water at about 50° C. or more, about 70° C. or more, or about 80 or more, and about 100° C. or less. At this time, also for uniform dispersion of a hyaluronic acid and hypromellose, a stirrer or the like may be used.

When a green tea catechin is used, a separate green tea catechin is added to the solution in which a hyaluronic acid and hypromellose are dispersed as described above, or a green tea catechin solution is prepared separately therefrom, and then is prepared in a two-component liquid form or a three-component liquid form.

In the case of preparing additive solution in a two- component liquid form or a three-component liquid form, the type of liquid forms or the solubility temperature may be set in consideration of the solubility characteristics of additional additive.

For example, in the case of an additive having high solubility in water at room temperature, an aqueous solution at room temperature may be prepared. In the case of an additive that is not highly soluble in water at room temperature, a separate aqueous solution or dispersion may be prepared in the form of dissolving the additive therein by preparing water at a high temperature. In the case of dissolving the additive in water at a high temperature, it may be used in combination with the solution for dispersing the hyaluronic acid and hypromellose.

When prepared as a two-component liquid form or a three-component liquid form as described above, a uniform mixture may be prepared by mixing the separately prepared additive liquid with the dispersion of a hyaluronic acid and hypromellose as described above under stirring.

When this is stirred at room temperature, the mixture change from a sol phase to a gel phase, but it does not change to a complete gel phase, and may exist in a half gel in the form of a fluid with high viscosity.

Next, the process of aging this half-gel-form mixture is performed. The aging process is a process of properly removing bubbles and an appropriate amount of water present in the half-gel-form mixture, putting the half-gel-form mixture in an airtight container, and leaving it at room temperature for about 1 to about 3 days. While left in the airtight container, the bubbles in the half-gel-form mixture escape, and an appropriate amount of water evaporates and forms on the upper portion of the airtight container. In order to efficiently remove these bubbles and evaporated water, it is also possible to adopt a method configured to observe whether water droplets form on the upper portion of the airtight container, properly open the lid to remove the water droplets from the upper portion, and then close the lid again.

After this aging process, the half-gel-form mixture has a higher viscosity and is shaped closer to a gel.

Such a gel may be processed into a sheet-type formulation such as a patch or a film through a drying process.

In this processing process, a release film may be used.

Specifically, using a coating equipment such as a bar coater, a spin coater, or an applicator, the gel-form mixture is uniformly applied on the release film. In consideration of the thickness after drying, the application thickness may be applied to about 2 times to about 5 times, or about 2 times to about 4 times the dry thickness.

After uniform application is made, it is transferred to a dryer to proceed with the drying process.

The drying may be performed by a method of dying for about 18 to about 30 hours under conditions of about 25 to about 45° C., preferably a method of drying for about 18 to about 30 hours at a temperature condition similar to human body temperature, or a method of rapidly drying at a high temperature of about 80 to about 120° C. for about 10 to about 60 minutes.

Through the drying process as described above, a food in the form of a sheet having a dry thickness of about ½ to about ¼ of the coating thickness may be prepared.

### EFFECT OF INVENTION

The edible sheet of the present disclosure can be processed into a thin form such as a sheet, has excellent adhesiveness to the mucous membrane in the oral cavity, has excellent durability, and is decomposed at an appropriate rate by moisture in the oral cavity, and can be used for various purposes.

### BRIEF DESCRIPTION OF THE DRAWINGS

FIG. 1 is a graph illustrating the durability evaluation results according to the zinc content of an edible sheet according to an embodiment of the present disclosure.

FIG. 2 is a graph illustrating the evaluation result of the adhesion force of the edible sheet according to an embodiment of the present disclosure.

FIG. 3 is a graph illustrating the durability evaluation results of the edible sheet according to an embodiment of the present disclosure.

FIGS. 4 and 5 are graphs illustrating the evaluation results of the degradability of the edible sheet according to an embodiment of the present disclosure, respectively.

FIGS. 6 and 7 are graphs illustrating the metal ion dissolution evaluation results of the edible sheet according to an embodiment of the present disclosure, respectively.

FIGS. 8 to 11 are graphs illustrating the characterization of EGCG/HPMC-based oral patch.

FIGS. 12 to 14 are graphs illustrating the rheological characterization of HPMC/HA/GTC oral patch.

FIGS. 15 to 17 are graphs illustrating the self-assemble ability of HPMC/HA/GTC oral patch via interaction with mucin.

FIGS. 18 and are graphs illustrating the mucoadhesiveness of HPMC/HA/GTC oral patch to resist against saliva flow.

FIG. 20 is a graph illustrating the sustained bactericidal effect of nanogel-formed HPMC/HA/GTC oral patch.

FIGS. 21 to 25 are graphs illustrating the biosafety of HPMC/HA/GTC oral patch.

### DETAILED DESCRIPTION

Hereinafter, the function and the effect of the invention are presented in more detail through specific examples of the invention. However, the following examples are only for illustrating the invention and the scope of the invention is not limited to or by them.

### EXAMPLES

### Preparation of Sheet (Patch) Type Food

### Example 1-1 (Without Zinc)

About 500 g of purified water was prepared and heated to about 100° C. 20 g of hypromellose (ES R&D Center, Hydroxypropylmethylcellulose) and 2 g of a hyaluronic acid (BLOOMAGE BIOTECHNOLOGY CORP, HA-F-016) were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 0.3 mm.

### Example 1-2

About 500 g of purified water was prepared, heated to about 100° C., and about 0.58 g of zinc sulfate was added thereto, and then completely dissolved by stirring.

20 g of hypromellose (ES R&D Center, Hydroxypropylmethylcellulose) and 2 g of a hyaluronic acid (BLOOMAGE BIOTECHNOLOGY CORP, HA-F-016) were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 0.3 mm.

### Example 1-3

About 500 g of purified water was prepared, heated to about 100° C., and about 5.76 g of zinc sulfate was added thereto, and then completely dissolved by stirring.

20 of hypromellose (ES R&D Center, Hydroxypropylmethylcellulose) and 2 g of a hyaluronic acid (BLOOMAGE BIOTECHNOLOGY CORP, HA-F-016) were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 0.3 mm.

### Example 1-4

About 500 g of purified water was prepared, heated to about 100° C., and about 57.6 g of zinc sulfate was added thereto, and then completely dissolved by stirring.

20 g of hypromellose (ES R&D Center, Hydroxypropylmethylcellulose) and 2 g of a hyaluronic acid (BLOOMAGE BIOTECHNOLOGY CORP, HA-F-016) were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 0.3 mm.

### Durability Evaluation

The edible sheet prepared in the above examples was cut into a rectangular shape of 5 cm in width and 0.5 cm in length to prepare a specimen.

About 0.5 cm of the central portion of the specimen was lightly wetted with distilled water, and then the water was wiped off.

Tensile strength was measured using a physical property analyzer (Yeonjin S-Tech, TXA texture analyzer).

Measurements were repeated 5 times in the same manner to obtain an average value.

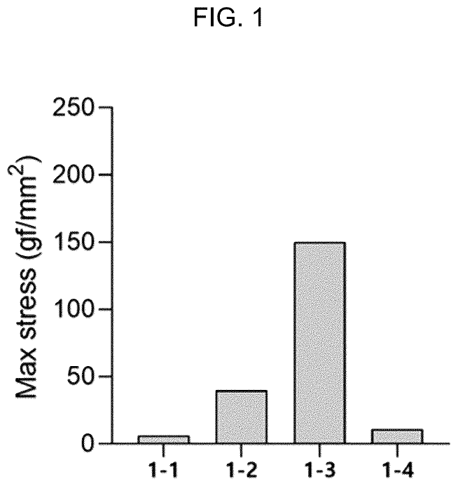

The measurement results are summarized in FIG. 1 below.

FIG. 1 is a graph illustrating the durability evaluation results according to the zinc content of an edible sheet according to an embodiment of the present disclosure.

Referring to FIG. 1, it was clearly identified that the tensile strength value increased from a minimum of two times to a maximum of several tens of times as zinc ions are added. In particular, in the case of Examples 1-3, it was clearly identified that the strength was significantly improved compared to other examples in which zinc ions are added.

As discussed above, this improvement in tensile strength is thought to be due to electrostatic interactions with metal ions and numerous hydroxy groups present in hyaluronic acid and hypromellose.

### Preparation of Sheet (Patch) Type Food

### Example 2-1 (Addition of Zinc and Other Additives)

250 g of purified water, 3.28 g of xylitol and 0.75 g of acesulfame potassium were added, and completely dissolved at room temperature (first liquid).

Separately, 250 g of purified water was heated to about 10020 C., and 24 g of glycerin (ES R&D Center), 1 g of catechin (Healing Co. Ltd., catechin 24), 3 g of L-menthol (Tien Yuan Chemical (PTE) LTD.), and 11.52 g of zinc sulfate (Serin Food Ingredients) were added thereto, and then completely dissolved by stirring (second Liquid).

20 g of hypromellose (ES R&D Center, Hydroxypropylmethylcellulose) and 2 g of a hyaluronic acid (BLOOMAGE BIOTECHNOLOGY CORP, HA-F-016) were added to the second solution, and dispersed through stirring.

The first solution and the second solution were mixed, placed in a stainless tray, closed with a lid, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 0.3 mm.

### Examples and Comparative Examples

In Example 2-1, a sheet (patch) type food was prepared in the same manner as in Example 2-1, except that the contents of hypromellose and hyaluronic acid were different.

The content is summarized in Table 1 below.

TABLE 1

| | Hypromellose (g) | hyaluronic acid (g) |
|---|---|---|
| Example 2-1 | 21 | 1 |
| Example 2-2 | 20 | 2 |
| Example 2-3 | 19 | 3 |
| Example 2-4 | 18 | 4 |
| (Comparative | | |
| Examples 1) | | |
| Example 2-5 | 17 | 5 |
| (Comparative | | |
| Examples 2) | | |
| Example 2-6 | 16 | 6 |
| (Comparative | | |
| Examples 3) | | |
| Example 2-7 | 15 | 7 |
| (Comparative | | |
| Examples 4) | | |
| Example 2-8 | 14 | 8 |
| (Comparative | | |
| Examples 5) | | |

### Adhesion Force Evaluation

For the sheet (patch) type foods prepared in the examples and comparative Examples, the adhesion force in the oral cavity was evaluated by the following method.

First, in order to create an experimental environment similar to the soft tissue in the oral cavity in terms of adhesion force, subcutaneous tissue of a pig was prepared.

Pig subcutaneous tissue was cut into a rectangular shape with a width of 1.5 cm and a length of 10 cm, and two pieces thereof were prepared.

The fat portion of the pig subcutaneous tissue was scraped with a blade to remove the fat layer, and the scraped surface was wiped with 80 wt % ethanol.

The edible sheet prepared in the Examples and Comparative Examples was cut to the same size as the pig subcutaneous tissue, lightly wetted with distilled water, placed between the two pieces of pig subcutaneous tissue, and then the two pieces of pig subcutaneous tissue were overlapped without applying any pressure other than gravity, and placed on the floor and joined.

Using a physical property analyzer (Yeonjin S-Tech, TXA texture analyzer), the maximum load applied at this time was measured while pulling the two overlapping pig subcutaneous tissues to both sides and removing the same.

Measurements were repeated 5 times in the same manner to obtain an average value.

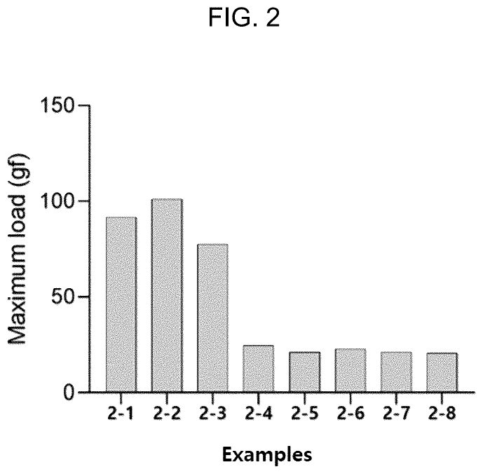

The measurement results are summarized in FIG. 2 below.

FIG. 2 is a graph illustrating the evaluation result of the adhesion force of the edible sheet according to an embodiment of the present disclosure.

Referring to FIG. 2, it was identified that the edible sheet according to an embodiment of the present disclosure has very good adhesion force to the human body. In particular, in the case of Examples 2-1 to 2-3, in which the content of a hyaluronic acid is relatively low, the maximum load required for separation from the pig subcutaneous tissue is about twice or more, compared to other examples, indicating that the adhesion strength was very good.

### Durability Evaluation

The edible sheet prepared in the above examples and comparative examples was cut into a rectangular shape of 5 cm in width and 0.5 cm in length to prepare a specimen.

About 0.5 cm of the central portion of the specimen was lightly wetted with distilled water, and then the water was wiped off.

Using a physical property analyzer (Yeonjin S-Tech, TXA texture analyzer), the stress-strain was measured until the specimen broke while pulling both sides of the specimen, and the breaking strength thereof was measured.

Measurements were repeated 5 times in the same manner to obtain an average value.

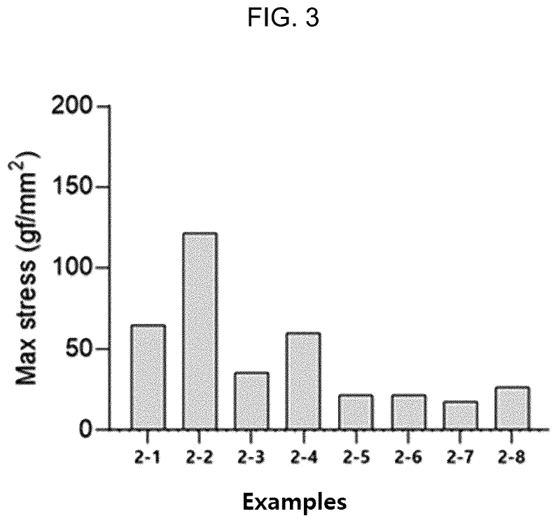

The measurement results are summarized in FIG. 3 below.

FIG. 3 is a graph n illustrating the durability evaluation results of the edible sheet according to an embodiment of the present disclosure.

Referring to FIG. 3, it was identified that the edible sheet according to an embodiment of the present disclosure was not easily broken even after being wetted with water because of the high breaking strength thereof. In particular, in the case of Examples 2-1 to 2-4, in which the content of a hyaluronic acid is relatively small, the breaking strength value was very high compared to other examples, indicating that the durability thereof was very good.

### Degradability Evaluation

The edible sheet prepared in the examples and comparative examples was cut into a rectangular shape of 1.5 cm in width and 1 cm in length to prepare three specimens.

The specimen and 3 ml of phosphate buffer were placed in a conical tube, the lid was closed, and then stored in an incubator at 37° C.

After 30 minutes, 1 hour, 2 hours, 4 hours, 8 hours, 16 hours, 32 hours, 64 hours, and 96 hours, 1 ml of the solution in each conical tube was extracted and stored in a petri dish, and was again supplemented with 1 ml of phosphate buffer in a conical tube.

The petri dish was placed into a dryer, dried, and the weight was measured, and the weight of the patch dissolved in the phosphate buffer aqueous solution was measured.

Measurements were repeated 5 times in the same manner to obtain an average value.

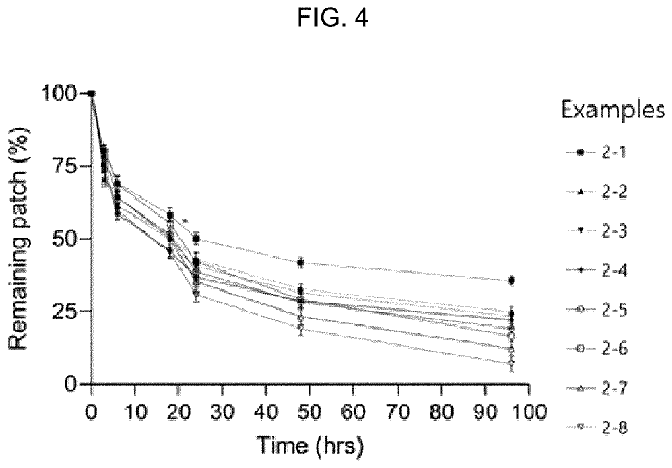

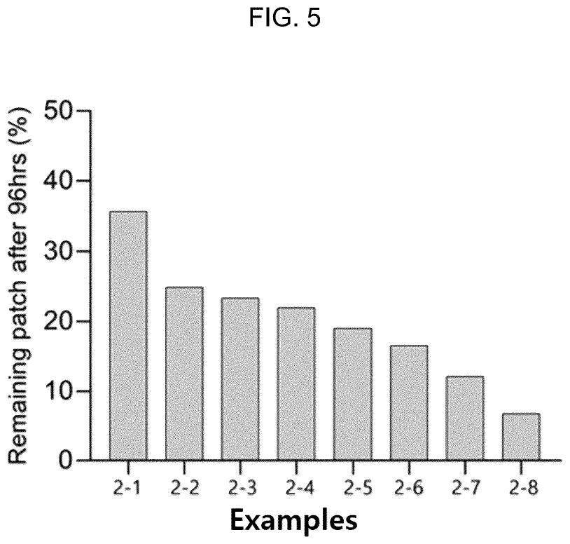

The measurement results are summarized in FIGS. 4 and 5 below.

FIGS. 4 and 5 are graphs illustrating the evaluation results of the degradability of the edible sheet according to an embodiment of the present disclosure, respectively.

Referring to FIG. 4, it was identified that it was most uniformly dissolved when the HPMC: HA ratio was 21:1. Referring to FIG. 5, it was identified that when the HPMC: HA ratio was 21:1, the largest amount remained, even after 96 hours, and the higher the HA ratio, the faster the erosion occurred.

### Zinc Dissolution Evaluation

The edible sheet prepared in the examples and comparative examples was cut into a rectangular shape of 5 cm in width and 0.5 cm in length to prepare a specimen.

The specimen and 3 ml of phosphate buffer were placed in a conical tube, the lid was closed, and then stored in an incubator at 37° C. (the experiment was carried out by preparing 10 identical specimen each.)

Samples were taken out one by one over time, and the concentration of eluted zinc in the specimen was measured.

Measurements were repeated 5 times in the same manner to obtain an average value.

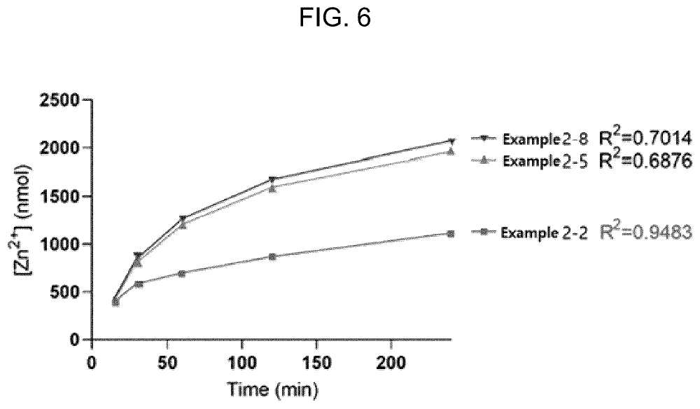

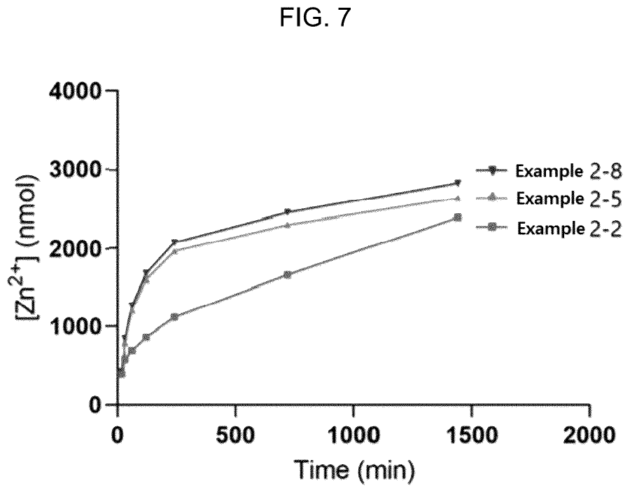

The measurement results are summarized in FIGS. 6 and 7 below.

FIGS. 6 and 7 are graphs illustrating the metal ion dissolution evaluation results of the edible sheet according to an embodiment of the present disclosure, respectively.

Referring to FIGS. 6 and 7, it was clearly identified that in the edible sheet according to an embodiment of the present disclosure, the release rate of the other components included in the sheet may be controlled according to the ratio of the hydrogel during the preparation of the sheet.

In particular, in the case of Example 2-2, the R² value is about 0.95, which is very close to 1, and it was identified that the components included in the sheet are released almost uniformly according to the time proportion under conditions similar to those of the human body.

### Preparation of Sheet (Patch) Type Food

### Example 3-1 (Addition of Green Tea Catechin)

250 g of purified water, 3.28 g of xylitol was added, and completely dissolved at room temperature (first liquid).

Separately, 250 g of purified water was heated to about 100° C., and 0.3 g of green tea catechin (Healing Co. Ltd., GTC) was added thereto, and then completely dissolved by stirring (second Liquid).

20 g of hypromellose (ES R&D Center, Hydroxypropylmethylcellulose) and 2 g of a hyaluronic acid (BLOOMAGE BIOTECHNOLOGY CORP, HA-F-016) were added to the second solution, and dispersed through stirring until a uniform slurry is formed.

The first solution and the second solution were mixed, placed in a stainless tray, closed with a lid, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-DO84), and dried at about 100° C. for about 30 minutes to obtain a sheet (patch) type food.

The dry thickness was found to be about 100 μm.

### Examples and Comparative Examples

In Example 3-1, a sheet (patch) type food was prepared in the same manner as in Example 3-1, except that the contents of green tea catechin was different.

The content is summarized in Table 2 below.

TABLE 2

| | Hypromellose | hyaluronic | Green tea |
|---|---|---|---|
| | (g) | acid (g) | catechin (g) |
| Example 3-1 | 20 | 2 | 0.3 |
| (HPMC/HA/1% GTC) | | | |
| Example 3-2 | 20 | 2 | 1 |
| (Comparative | | | |
| Examples 6) | | | |
| (HPMC/HA/3% GTC) | | | |

### Materials and Methods

### LC-MS/MS Analysis

Catechins in Green tea catechin were analyzed using an Agilent 1290 Infinity II UHPLC system coupled to a 6470 Triple Quadrupole MS (Agilent Technologies, USA) operated in both positive and negative ESI modes with dynamic multiple reaction monitoring (dMRM).

Chromatographic separation was performed using an ACQUITY UPLC BEH C18 column (1.7 μm, 2.1×100 mm; Waters, USA) at 35° C. The mobile phase consisted of (A) water and (B) acetonitrile, both containing 0.1% formic acid, at a flow rate of 0.34 mL/min. The gradient elution was as follows: 5% B (0-1 min), 5-50% B (1-5 min), 50% B (5-7 min), 50-80% B (7-9 min), 80-100% B (9-10 min), 100% B (10-14 min), and 5% B re-equilibration (14.1-18 min). The injection volume was 3 μL, and autosampler temperature was maintained at 7° C.

Calibration curves were constructed from 5 to 5000 ng/mL using DMSO-dissolved stock solutions (100 μg/mL) serially diluted in 75% methanol+0.1% BHT. Data were processed with MassHunter Workstation (vB. 07.00, Agilent Technologies).

### UV-Vis Spectrophotometry

0.2 mg of green tea catechin, and HPMC (hypromellose)/HA (hyaluronic acid)/GTC (green tea catechin) composite gels containing catechin at 0.2 mg/mL was diluted 10-fold in distilled water and analyzed using a UV-Vis spectrophotometer (Shimadzu, Japan).

### Fourier Transform Infrared (FT-IR) Spectroscopy

Samples of EGCG (Sigma-Aldrich, USA), HPMC, and EGCG (epigallocatechin-3-gallate)/HPMC composites were hydrated in deionized water and lyophilized. The FT-IR spectra were acquired using an FT-IR spectrometer (PerkinElmer, USA).

### Scanning Electron Microscopy (SEM)

Freeze-dried oral patches and leaves from Camellia sinensis (Green tea) were imaged using SEM. Additionally, rehydrated hydrogels were prepared by immersing the dry patches in PBS or saliva, followed by lyophilization and SEM imaging. Hydration Assay and Rheological Analysis

HPMC/HA, HPMC/HA/1% GTC, and HPMC/HA/3% GTC patches were treated with PBS or saliva and imaged over time using the FOBI imaging system (NeoScience, South Korea). 0.25 g of each patch samples were immersed in 2 mL of PBS or saliva, and their viscoelastic properties were evaluated using a rheometer (TA instruments, USA) across a frequency range of 1-10 Hz. For cyclic strain testing, HPMC/HA/3% GTC patches were exposed to 0% and 1000% strain to assess rheological recovery.

### Nanogel Formation Assay

Hydrated hydrogels (HPMC/HA and HPMC/HA/GTC) were prepared at a concentration of 2 mg/mL in PBS, saliva, or 1.19 mg/mL of bovine submaxillary mucin (Sigma-Aldrich, USA), then diluted 1:40 for imaging. For turbidity kinetics, a 1:10 dilution was monitored at 90-second intervals using a UV-Vis spectrophotometer (Shimadzu, Japan).

### In Vivo Biostability

Eight-week-old ICR mice were anesthetized with 100mg/kg of ketamine and 16.7 mg/kg of xylazine cocktail. 10 mg of the oral patch was applied to the tongue. 0.2 mg/kg of Pilocarpine was administered intraperitoneally to stimulate salivation. Tongue tissues were harvested at 30 and 60 minutes post-injection for SEM and fluorescence imaging.

### In Vitro Bactericidal Assay

To evaluate antibacterial activity, bovine submaxillary mucin (1.19 mg/mL) was mixed with GTC or 2 mg of the oral patch and applied to surface-treated culture slides. After 5 minutes, the slides were vigorously washed with PBS, inoculated with Porphyromonas gingivalis, and incubated for 24 hours. Optical density at 600 nm was measured, and fluorescence staining was performed using Hoechst 33342 and WGA-FITC, followed by confocal microscopy.

### Biosafety Evaluation

For in vitro cytotoxicity, MG-63 cells (5×10⁴) were seeded on the bottom of a Transwell plate. 3.2 mg of oral patch was placed in the insert and incubated for 24 hours. Cell viability was assessed using the CCK-8 assay (Dojindo, Japan) at 450 nm, and live/dead staining was performed using calcein-AM and propidium iodide.

For in vivo biosafety, Eight-week-old ICR mice were used. Mice were anesthetized with 100 mg/kg of ketamine and 16.7 mg/kg of xylazine cocktail. 10 mg of the oral patch was applied to the oral cavity of 8-week-old ICR mice and allowed to disperse naturally. Body weight was monitored for 14 days, after which the mice were euthanized. The tongue, liver, spleen, kidney, and lungs were harvested and processed for H&E staining.

### Histological Analysis

Formalin-fixed, paraffin-embedded tissue sections were stained with hematoxylin and eosin (H&E) following standard procedures. Briefly, sections were deparaffinized in xylene and rehydrated through a graded ethanol series. Nuclear staining was performed using Harris hematoxylin (7 min), followed by differentiation in 1% hydrochloric acid and bluing in running tap water. Counterstaining was done with eosin (3 s), and sections were dehydrated through ascending ethanol grades and cleared in xylene. Slides were mounted with a coverslip and imaged at 400× magnification using a slide scanner (Aperio AT2, Leica, Germany).

### Clinical Study

50 healthy adult volunteers enrolled, 12 were excluded based on pre-defined criteria. The remaining 38 participants were randomly assigned to a control or treatment group. Each group received a placebo patch or a catechin-containing patch, respectively, with instructions to apply the patch once 10 minutes before sleep, for 14 days. Unstimulated saliva was collected before and after the intervention. Oral bacterial load was quantified using RT-PCR.

### Statistical Analysis

All data were analyzed using GraphPad Prism 9 (GraphPad Software, USA). Statistical significance was evaluated using one-way ANOVA followed by Tukey's post hoc test, or Student's t-test, as appropriate. A p-value<0.05 was considered statistically significant.

### Physicochemical Characteristics of the HPMC/HA/GTC Oral Patch

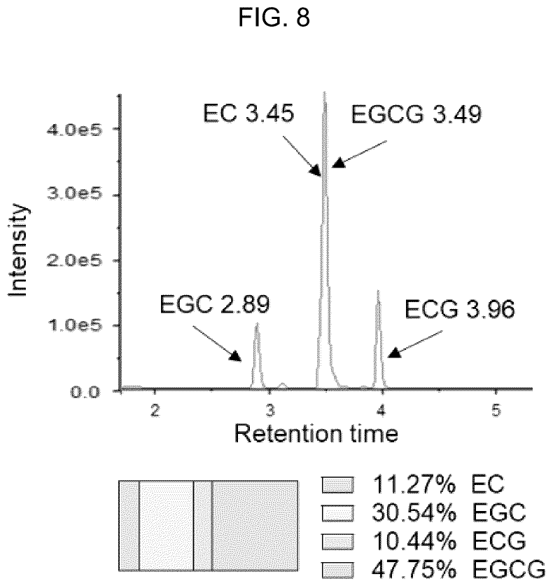

As a result of the catechin component analysis (LC-MS) of green tea catechin, referring to FIG. 8, the green tea catechin according to one embodiment of the present invention contained 11.27% epicatechin (EC), 30.54% epigallocatechin (EGC), 10.44% epicatechin gallate (ECG), and 47.75% epigallocatechin-3-gallate (EGCG).

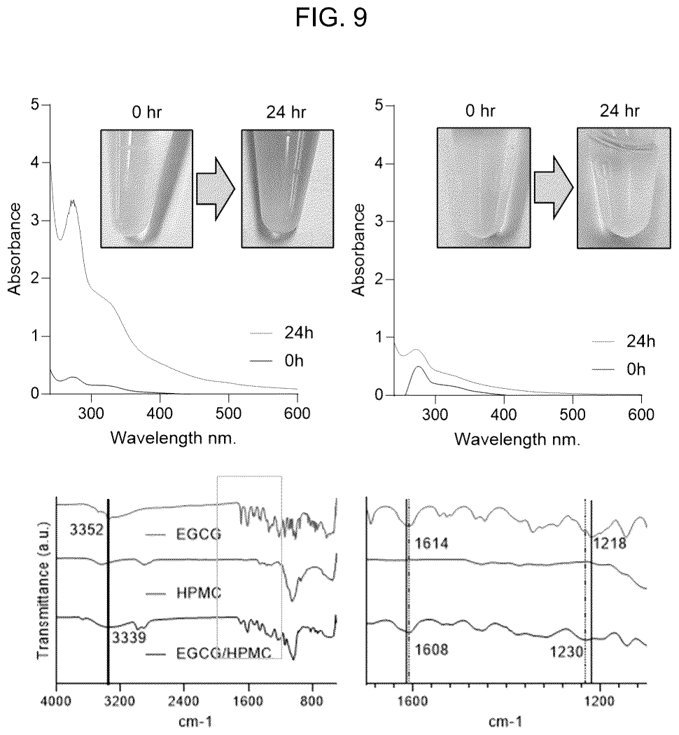

The potential of the solubilized green tea catechin extract to prevent premature oxidation was confirmed (UV-Vis). As shown in FIG. 9, a solubilized catechin extracts underwent rapid oxidation within 24 hours, evidenced by a brown coloration and a strong absorption peak at 280-340 nm. In contrast, extracts encapsulated within the HPMC/HA mixture exhibited minimal oxidation peaks at 280-340 nm even after 24 hours. This polymeric encapsulation system enabled the preparation of thin, slowly air-dried patches without premature oxidation of catechins, providing a significantly more cost-effective alternative to freeze-drying methods.

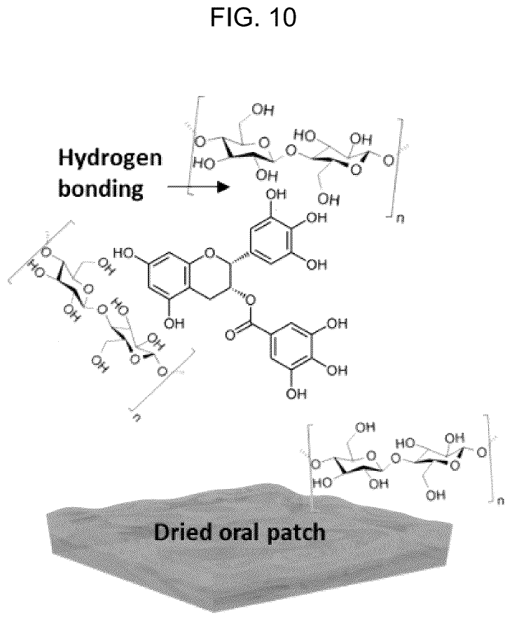

After hydrating and freeze-drying samples of epigallocatechin-3-gallate (EGCG), hypromellose (HPMC), and the EGCG/HPMC composite, FT-IR spectra were measured. As shown in FIG. 10, hydrogen bonding between the hydroxyl group (—OH) of HPMC and the gallol group of EGCG was confirmed, suggesting a partially cross-linked network.

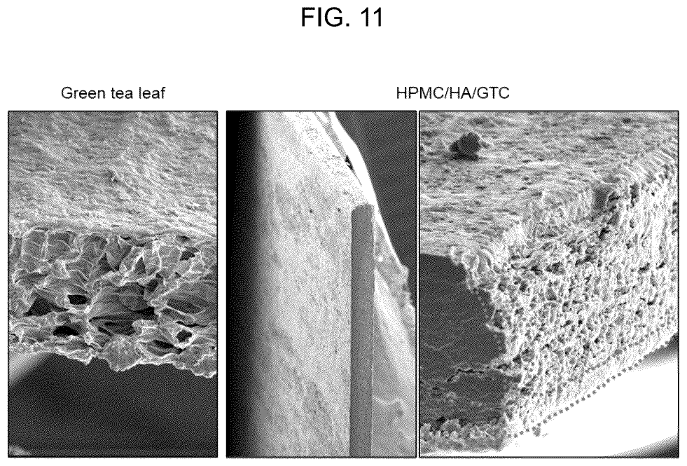

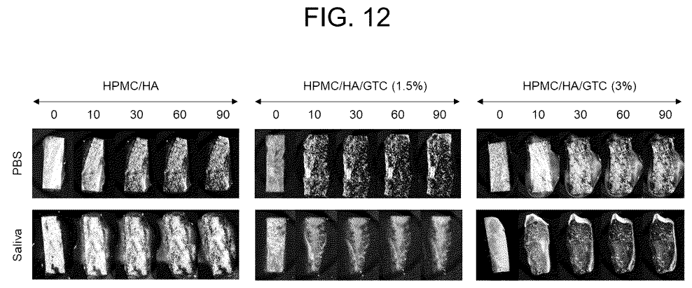

Finally, the air-dried oral patches and green tea leaves were imaged using SEM, as shown in FIG. 11, the final air-dried patches exhibited uniform thinness (100˜200 μm) and densely packed peripheral layers 3, structurally mimicking the microstructure of green tea leaves.

### Changes in Rheological Properties of Patches by Saliva

Dried patches composed of HPMC/HA, HPMC/HA/1.5% GTC, and HPMC/HA/3% GTC were hydrated with phosphate-buffered saline (PBS) or saliva. As shown in FIG. 12, the HPMC/HA patches showed similar degradation kinetics, retaining their original shape after 90 minutes of hydration with either PBS or saliva. Similarly, patches containing HPMC/HA/1.5% GTC maintained structural integrity upon exposure to both PBS and saliva over 90 minutes. However, patches containing HPMC/HA/3% GTC rapidly dissolved into a transparent, colloid-like liquid within 10 minutes of contact with saliva, while remaining intact upon exposure to PBS.

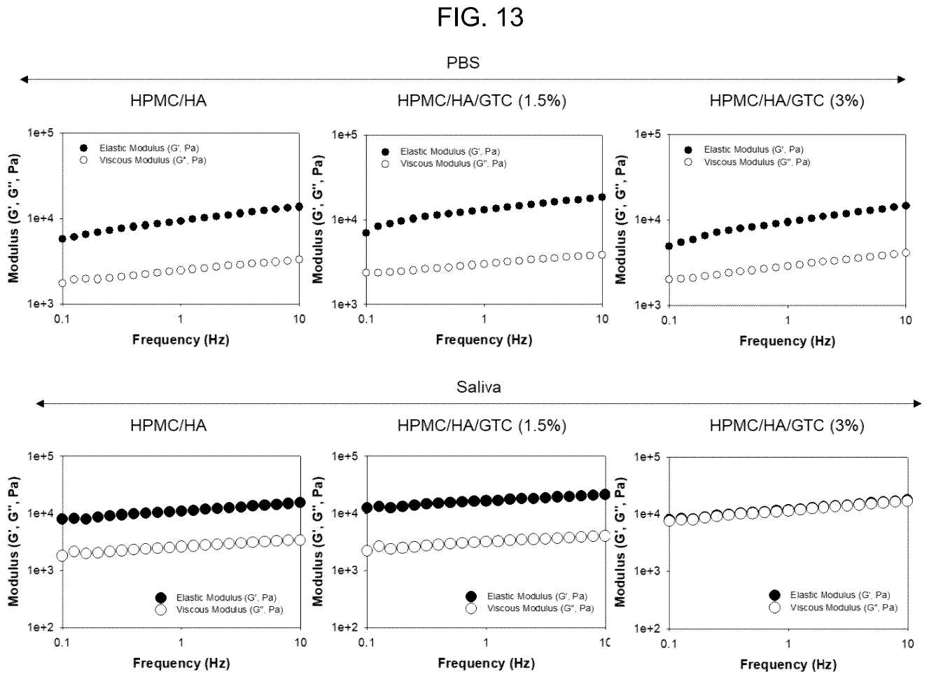

As a result of rheological analysis, as shown in FIG. 13, when exposed to PBS, all patches demonstrated stable viscoelastic properties (G′>G″) across frequencies ranging from 0.1 to 1 Hz, with elastic moduli between 9,000 and 10,000 Pa and viscous moduli between 3,000 and 4,000 Pa at 1 Hz. Upon exposure to saliva, HPMC/HA and HPMC/HA/1.5% GTC patches retained similar viscoelastic characteristics, with elastic moduli between 9,500 and 11, 000 Pa and viscous moduli between 3,000 and 4,000 Pa at 1 Hz. In contrast, the rheological properties of HPMC/HA/3% GTC patches drastically changed upon saliva exposure, with viscous modulus at 1 Hz increasing significantly to approximately 11,000 Pa, nearly equaling the elastic modulus (11,300 Pa), indicative of a hydrocolloid state between solution and gel.

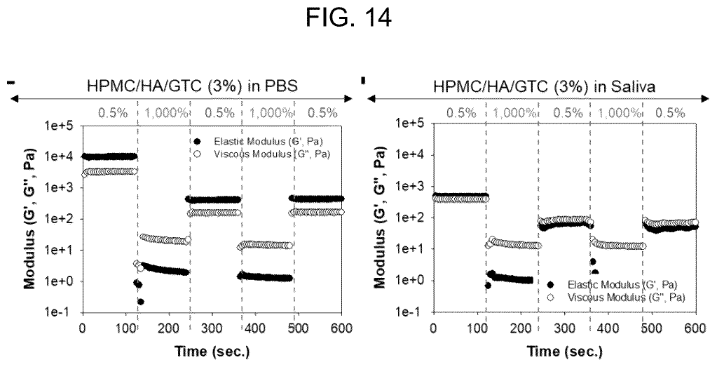

Finally, as shown in FIG. 14, alternative sweep analysis confirmed that these rheological changes resulted from saliva-induced polymer network remodeling rather than simple degradation.

### Saliva-Induced Nanoparticle Formation Mechanism

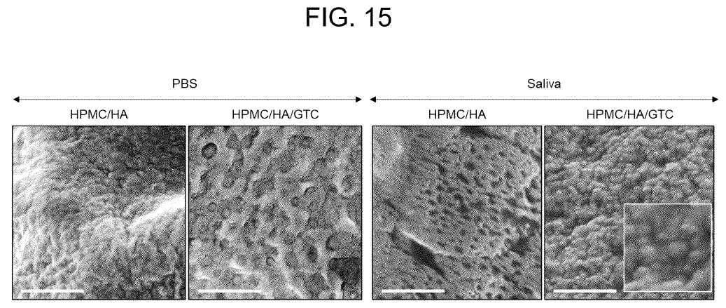

As shown in FIG. 15, SEM analysis of the microstructural changes of the patches immediately after hydration with PBS or saliva revealed that the HPMC/HA and HPMC/HA/3% GTC patches hydrated in PBS exhibited similar morphologies. In contrast, upon saliva exposure, the dense lamellar structure of HPMC/HA/3% GTC transformed into surfaces covered by uniform nanospheres (˜2 nm in diameter), while the HPMC/HA patches showed a porous structure typical of swelling hydrogels.

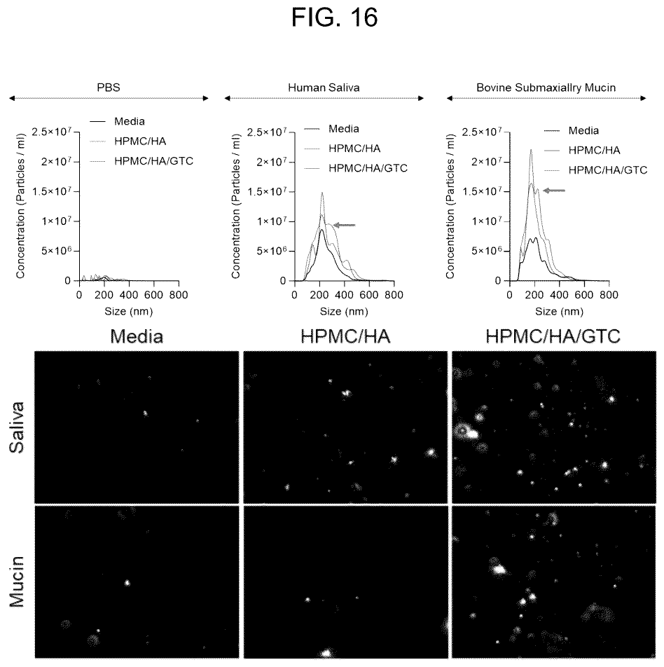

As shown in FIG. 16, NTA results revealed a distinct peak of nanoparticle formation around 300 nm when the HPMC/HA/3% GTC patch was dissolved in human saliva or purified bovine mandibular mucin solution. A greater number of nanoparticles were formed in the saliva-dissolved HPMC/HA/3% GTC.

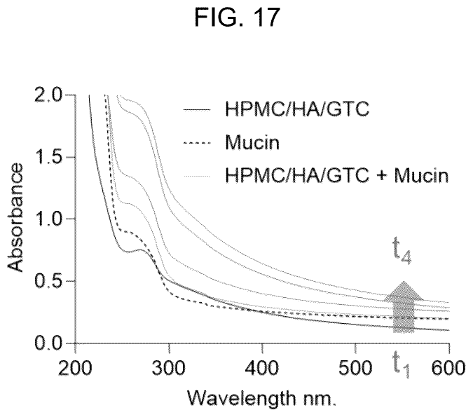

As shown in FIG. 17, UV-Vis spectroscopy further demonstrated time-dependent increases in absorbance at 500-600 nm upon mixing HPMC/HA/GTC with mucin, indicative of ongoing intermolecular interactions.

### In Vivo Mucoadhesiveness and Drug Delivery Potential Of HPMC/HA/GTC Oral Patch

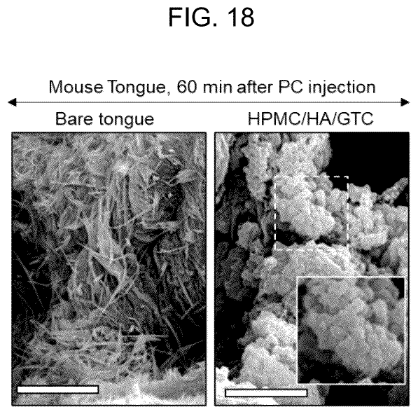

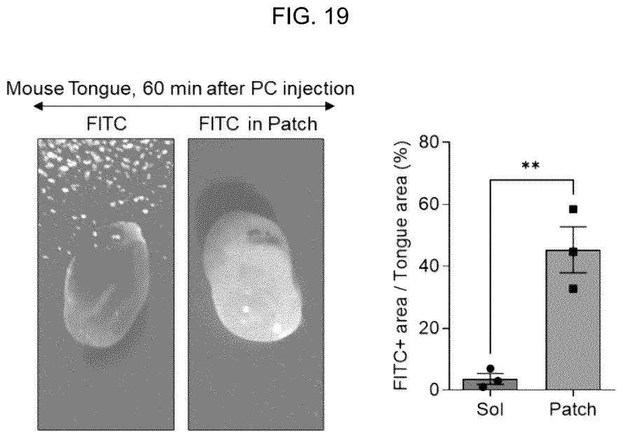

In vivo mouse tongue experiments, as shown in FIG. 18, SEM imaging after 60 minutes showed nanoparticles tightly adhered to tongue mucosa and filiform papillae despite high salivary flow rates, confirming strong oral tissue adhesion and successful in situ formation of nanoparticles.

To assess potential as drug delivery vehicles, as shown in FIG. 19, FITC-labeled bovine serum albumin (BSA) was loaded into HPMC/HA/GTC patches and subjected to high salivary flow conditions. FITC-BSA loaded in patches remained on the tongue surface after 60 minutes, covering an area twice the original patch size, whereas unencapsulated FITC-BSA was completely washed away.

These findings demonstrate the potential of saliva-induced nanoparticles for effective and sustained oral mucosal drug delivery.

### Bactericidal Activity and Antibiofilm Efficacy

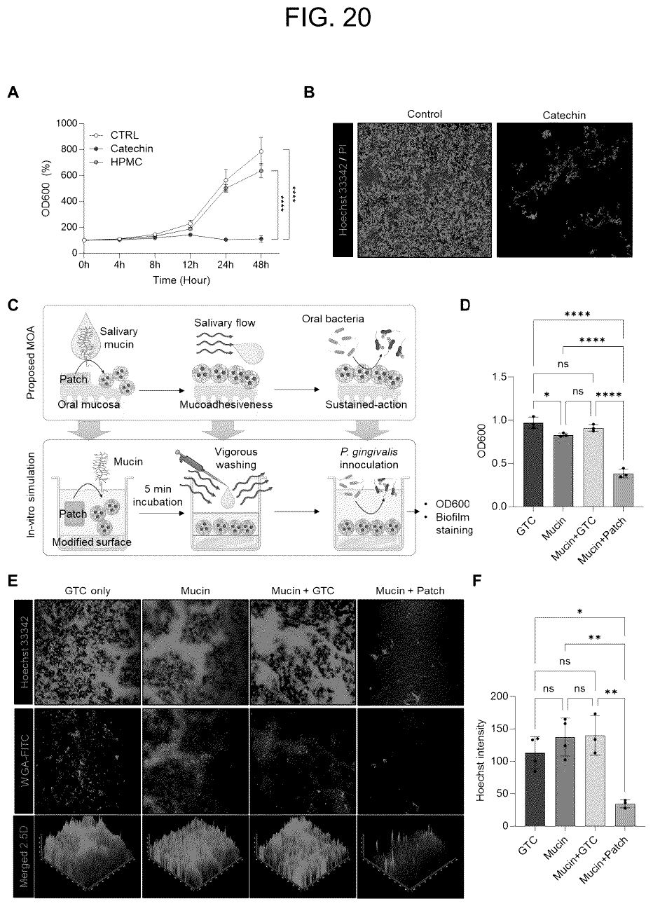

As shown in FIG. 20, the group treated with mucin and the HPMC/HA/GTC oral patch showed significantly reduced bacterial viability and biofilm formation compared to the other groups (GTC alone, mucin alone, and mucin & GTC).

Together, even in oral environments such as saliva flow, these findings demonstrate that the nanogel formed on the surface of the HPMC/HA/GTC oral patch effectively suppresses P. gingivalis growth through sustained release and surface adhesion.

### Biosafety of HPMC/HA/GTC Oral Patch

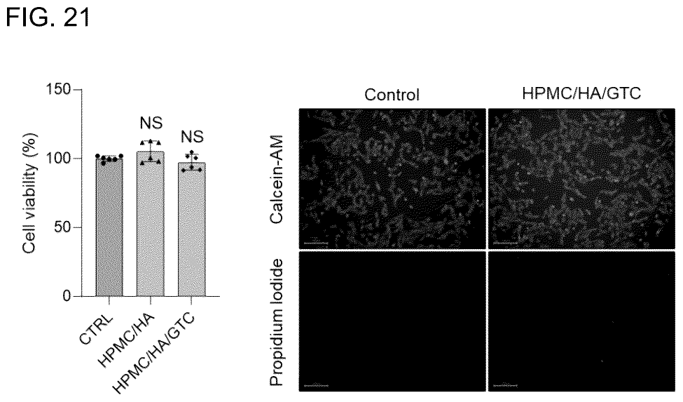

As shown in FIG. 21, in vitro cytotoxicity tests showed that Both HPMC/HA and HPMC/HA/GTC patches demonstrated no significant cytotoxicity in the CCK-8 assay, and Calcein AM/PI staining confirmed the absence of PI-positive (dead) cells.

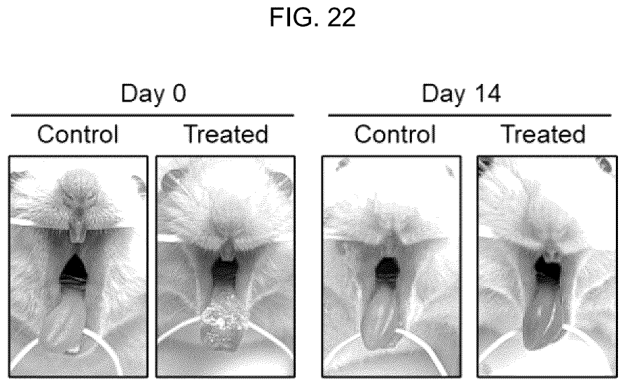

As shown in FIG. 22, Visual inspection revealed no redness, swelling, or visible irritation in the patch-treated groups.

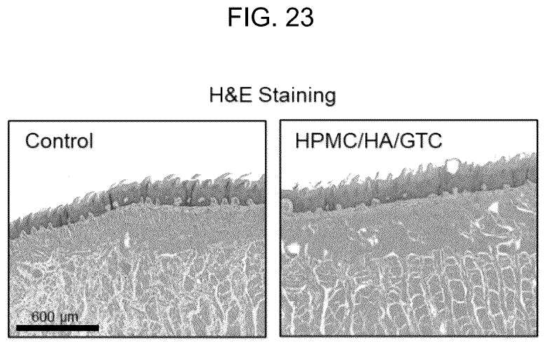

As shown in FIG. 23, Histological examination further confirmed the absence of lymphocytic infiltration or structural damage, including preservation of filiform papillae in patch-treated mouse tongues, indicating no mucosal irritation from repeated patch applications.

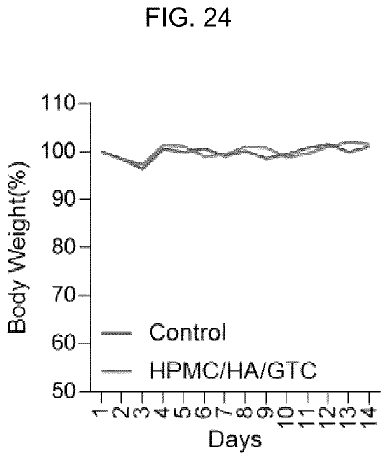

As shown in FIG. 24, no differences in body weight were observed between control and patch-treated mice, demonstrating no systemic toxicity from repeated patch consumption.

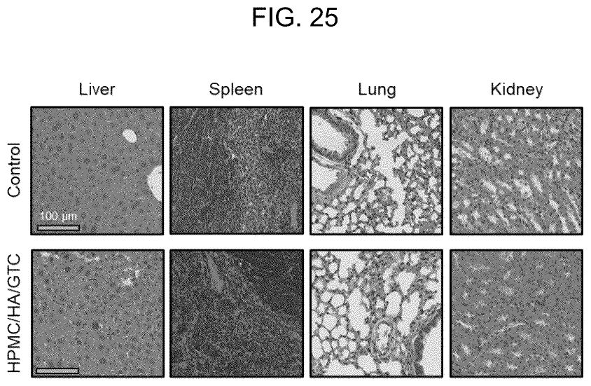

As shown in FIG. 25, histological analyses of major organs including liver, spleen, lung, and kidney revealed no signs of inflammation or fibrotic lesions in the patch-treated group, showing similar histological profiles compared to the controls.

In summary, the HPMC/HA/GTC patch exhibited excellent biosafety, demonstrating no local mucosal irritation or systemic toxicity under clinical application conditions.

Accordingly, it is considered that the edible sheet according to an embodiment of the present disclosure can be used not only as a simple food, but also as a health functional food containing a specific active ingredient, a patch for drug release, and the like.

1. An edible sheet including hyaluronic acid, hypromellose and green tea catechin.

2. The edible sheet according to claim 1, wherein the hyaluronic acid includes one or more of a cross-linked hyaluronic acid and a non-cross-linked hyaluronic acid.

3. The edible sheet according to claim 1, wherein the hypromellose has a methoxy substitution rate of 10 to 35%, and a hydroxypropyl substitution rate of 1 to 15%.

4. The edible sheet according to claim 1, wherein the edible sheet has a dry thickness of 0.05 to 1 mm.

5. The edible sheet according to claim 1, wherein the edible sheet includes 0.1 to 20 parts by weight of a hyaluronic acid and 3 to 7 parts by weight of a green tea catechin, based on 100 parts by weight of hypromellose.

6. The edible sheet according to claim 1, wherein the edible sheet forms multiple nanospheres with a diameter of 0.1 to 2 nm on the surface of the sheet, upon exposure to saliva.

7. The edible sheet according to claim 1, wherein the edible sheet prevents oxidation of green tea catechin.

8. The edible sheet according to claim 1, wherein the edible sheet is air-dried.

9. The edible sheet according to claim 1, wherein the hyaluronic acid has a weight average molecular weight of 10,000 to 1,000,000 g/mol.

10. The edible sheet according to claim 1, wherein the hypromellose has a weight average molecular weight of 100,000 to 1,000,000 g/mol.

11. The edible sheet according to claim 1, further including ions of one or more edible metals selected from the group consisting of zinc, copper, iron, nickel, manganese, chromium, calcium, magnesium, sodium, potassium and selenium.

12. The edible sheet according to claim 11, wherein the hyaluronic acid and the hypromellose are in the form of a complex coordinated around the metal.

13. A method of preventing oxidation of green tea catechin in the edible sheet according to claim 1, the method comprising:
preparing the edible sheet comprising hyaluronic acid, hypromellose, and green tea catechin, and
encapsulating the green tea catechin within a polymeric encapsulation system comprising hyaluronic acid and hypromellose, thereby inhibiting premature oxidation of the green tea catechin.
